(19) United States
(12) Patent Application Publication
(10) Pub. No.: US 2012/0178964 A1
(43) Pub. Date: Jul. 12, 2012

(54) Catalyst, Use Thereof and Process of Hydrogenating Aryl Aldehydes

(75) Inventors: Philip O. Nubel, Naperville, IL (US); Thomas M. Bartos, Naperville, IL (US); Sameer Talreja, Lombard, IL (US); Stephen P. Brugge, Naperville, IL (US)

(73) Assignee: BP CORPORATION NORTH AMERICA INC, NAPERVILLE, IL (US)

(21) Appl. No.: 13/496,070

(22) PCT Filed: Sep. 20, 2010
(86) PCT No.: PCT/US10/49477

§ 371 Date: Mar. 14, 2012

Related U.S. Application Data
(60) Provisional application No. 61/247,416, filed on Sep. 30, 2009.

(51) Int. Cl. C07C 51/00

(52) U.S. Cl. 562/473

(57) ABSTRACT

This invention provides a catalyst and the use thereof and a process for hydrogenating carboxyaryl aldehydes with selectivity to hydroxyalkylaryl monocarboxylic acids. The catalyst comprises iridium.

### FIELD OF THE INVENTION

This invention relates to a process, catalyst and use of catalyst for hydrogenating carboxyaryl aldehydes with selectivity to hydroxyalkylaromatic carboxylic acid and, in some embodiments, conversion of carboxybenzaldehyde to product comprising hydroxymethylbenzoic acid using a catalyst comprising iridium or rhodium, optionally in the presence of aromatic carboxylic acid.

### BACKGROUND OF THE INVENTION

Hydroxyalkylaryl monocarboxylic acids, such as p- and m-hydroxymethylbenzoic acid (pHMBA and mHMBA, respectively), are important raw materials for synthesis of corresponding homopolymer polybenzoates and poly(p-methylene benzoate), and for copolymerization with lactams as in U.S. Pat. No. 4,528,361. U.S. Pat. No. 4,448,987 discloses production of hydroxymethylaryl monocarboxylic acids by selective hydrogenation of aryldicarboxylic acids using a rhenium catalyst. Hydroxymethylbenzoic acids also are produced as by-products in the oxidation of xylenes, such as para- or meta-xylene, to aromatic carboxylic acids such as tere- and isophthalic acids, which are raw materials for manufacture of polyethylene terephthalate and copolyesters used for fiber, packaging and molding resin applications.

U.S. Pat. No. 3,584,039 discloses that pHMBA is obtained in combination with p-toluic acid (pTOL) in the purification of crude or impure terephthalic acid products containing 4-carboxybenzaldehyde (4CBA) by contacting aqueous solutions thereof with hydrogen in the presence of a Group VIII metal catalyst at elevated temperature and pressure. 4CBA is hydrogenated to pHMBA, which is converted to pTOL by hydrogenolysis. U.S. Pat. No. 4,933,492 discloses hydrogenating impure isophthalic acid containing 3-carboxybenzaldehyde (3CBA), such as is obtained by oxidation of feed materials comprising or derived from meta-xylene, to mHMBA and m-toluic acid (mTOL).

Chinese Patent Applications 200710047875.7 and 2007100439350.2 (Publication Nos. CN 101428226A and CN 101347737A) disclose active carbon- or titania-supported palladium and ruthenium, nickel, zinc or copper as catalyst for purifying terephthalic acid contaminated with 4CBA by selectively hydrogenating 4CBA to HMBA with reduced hydrogen consumption. U.S. Pat. No. 4,260,817 describes purification of terephthalic acid having aldehyde impurities, such as 4CBA, by hydrogenation of aldehyde substituents to hydroxymethyl groups and in turn to alkyl groups (e.g., 4CBA→pHMBA→pTOL) using carbon-supported catalyst containing two or more of palladium, platinum, rhodium, ruthenium, osmium and iridium. The catalyst is said to push conversion of hydroxymethyl groups to alkyl groups.

An improved process for producing hydroxyalkylaryl monocarboxylic acids would be desirable, as would such a process having applicability in the purification of aromatic carboxylic acids.

### SUMMARY OF THE INVENTION

This invention provides for improved production of hydroxyalkylaromatic monocarboxylic acids by hydrogenation of carboxyaryl aldehydes in the presence of a catalyst which is selective for hydrogenation of aldehyde substituents to hydroxyl moieties. Hydroxyalkylaryl monocarboxylic acid produced according to embodiments of the invented process preferably exceeds that using known catalysts, other things being equal and in embodiments is generated in excess of alkylaryl monocarboxylic acid. Selectivity also is such that conversion of carboxyl moieties to alkyl is disfavored and, accordingly, hydrogenation of carboxyaryl aldehydes with selectivity to hydroxyalkyaryl monocarboxylic acids proceeds without substantial conversion of aromatic carboxylic acids if present. Separation of hydroxyalkylaryl monocarboxylic acid from the reaction mixture, including hydrogenation by-products and aromatic carboxylic acids that may be present, is facilitated by differences in solubilities of the hydroxyalkyaryl acid and other species such as corresponding aromatic acids and alkylaryl monocarboxylic acids. Differences in co-crystallization of the hydrogenated products with aromatic carboxylic acids such as terephthalic acid or isophthalic acid also contribute to improved separations in embodiments of the invention in which aromatic carboxylic acid is present.

In one embodiment, the invention provides a process for producing an hydroxyalkylaromatic monocarboxylic acid comprising contacting a feed comprising a carboxyaryl aldehyde with hydrogen in the presence of catalyst comprising iridium or rhodium to form a product comprising hydroxyalkylaryl monocarboxylic acid. In preferred embodiments, the catalyst additionally comprises palladium and/or contacting the feed with hydrogen in presence of the catalyst is conducted in the presence of aromatic carboxylic acid. Embodiments of the invention in which the catalyst comprises palladium in addition to iridium or rhodium provide greater selectivity to hydroxyalkylaromatic monocarboxylic acid than when catalyst containing only palladium is used, other things being equal.

In more specific embodiments the invention provides a process for producing hydroxymethylbenzoic acid comprising contacting a feed comprising carboxybenzaldehyde with hydrogen in the presence of a catalyst comprising iridium or rhodium, and preferably palladium in addition to iridium or rhodium, to form a product comprising hydroxymethylbenzoic acid. In embodiments in which the catalyst comprises iridium or rhodium and palladium, selectivity to hydroxymethylbenzoic acid exceeds that of catalysts with palladium, iridium or rhodium alone, other things being equal.

Embodiments of the invention also provide such processes in which aromatic carboxylic acid is present during contacting carboxyaryl aldehyde with hydrogen. Preferably, carboxyaryl aldehyde is converted in such a process with selectivity to hydroxyalkyaryl monocarboxylic acid and without substantial loss of aromatic carboxylic acid to alkylaromatic or ring hydrogenated species. Aromatic carboxylic acid can be present as part of a carboxyaryl aldehyde-containing feed for the process or from other sources.

In another embodiment, the invention provides a process for manufacture of an aromatic carboxylic acid comprising contacting a feed comprising aromatic carboxylic acid and impurities comprising at least one aromatic aldehyde with hydrogen in the presence of a catalyst comprising iridium or rhodium to form a product comprising aromatic carboxylic acid and hydroxyalkylaromatic monocarboxylic acid with improved selectivity thereto. In some embodiments, selective hydrogenation according to the invention is applied in the purification of impure aromatic carboxylic acid containing arylaldehyde impurities, such as impure or crude terephthalic or isophthalic acid (“TA” or “IA”) such as made by oxidation of p- or m-xylene or their partially oxidized derivatives or otherwise containing aldehyde impurities such as carboxybenzaldehydes, aromatic dialdehyde or both, and in particular crude TA and impurities comprising 4CBA such as is obtained by oxidation of feedstock comprising p-xylene or partially oxidized derivative(s) thereof or combinations, by hydrogenation of aldehyde impurity to pHMBA with improved selectivity thereto.

Processes according to the invention also include separation of hydroxyalkyaryl monocarboxylic acid from reaction product mixtures, preferably by solid-liquid separation techniques wherein a liquid phase comprising hydroxyalkylaryl monocarboxylic acid, preferably in excess of alkylaryl monocarboxylic acid, is separated from a solid phase product. Separation by such techniques is facilitated by greater solubilities of hydroxyalkylaryl monocarboxylic acids than alkylaryl monocarboxylic acids or aromatic carboxylic acids in solvents compatible with other process manipulations or steps. The invention includes embodiments in which alkylaryl monocarboxylic acid levels are low enough that separation thereof is simplified or unnecessary, thereby enabling utility of simplified separation equipment, reduced pressures or other milder conditions for separations.

Catalysts according to or used according to the invention comprise iridium or rhodium. Preferred catalysts for some embodiments additionally comprise palladium. Supported catalysts are preferred when contacting feed comprising carboxyaryl aldehyde with hydrogen in the presence of aromatic carboxylic acid. Catalysts comprising iridium and palladium supported on a particulate support material are preferred in such embodiments.

### DETAILED DESCRIPTION OF THE INVENTION

In greater detail, the invention provides a process, catalyst and use of catalyst for conversion of aryl aldehydes to hydrogenated derivatives with selectivity to hydroxyalkylaryl monocarboxylic acids. In addition, hydrogenolysis of carboxyl groups and ring hydrogenation of aromatic nuclei are disfavored in comparison to hydrogenation of the aldehyde moieties of the starting carboxyaryl aldehyde such that the hydrogenation in the presence of aromatic carboxylic acids can be conducted without adverse affect to the acids. In this regard, it will be understood that unless otherwise indicated by context, the term aromatic carboxylic acid as used herein refers to aromatic carboxylic acid lacking substituents other than carboxylic acid groups.

Different solubilities in aqueous and other solvents of the hydroxyalkylaryl monocarboxylic acid and other products or compounds present in a reaction mixture from the invented process can afford opportunities for facilitating or improving separations and recoveries of the hydroxyalkylaryl monocarboxylic acid products or other species. Application of the invention in processes for manufacture or purification of aromatic carboxylic acids from carboxyaryl aldehyde impurity-containing feed materials can provide improved separation of hydrogenated derivatives of the carboxyaryl aldehyde as compared with conventional processes. Lower pressure separations are facilitated in some embodiments, affording process flexibility and simplified separation techniques and equipment.

Carboxyaryl aldehyde starting materials suitable for use according to the invention comprise an aromatic nucleus substituted with carboxylic acid and aldehyde groups. Specific examples include carboxybenzaldehydes such as 2-carboxybenzaldehyde, 3CBA, 4CBA, dicarboxybenzaldehydes (e.g., 2,4-, 2,5- and 3,4-dicarboxybenzaldehyde) and the 3,4-anhydride, and carboxynaphthaldehydes. The carboxyaryl aldehyde feed or starting materials can be in pure or relatively pure form or it can comprise other aromatic species such as aromatic carboxylic acids and partial oxidation products of alkyl-substituted arenes in which carboxyaryl aldehyde is present in only minor or fractional weight percent amounts.

The carboxyaryl aldehyde-containing starting material is contacted with hydrogen in the presence of catalyst comprising iridium or rhodium according to the invention. Contacting preferably is conducted with the aldehyde starting material present in any suitable form. Preferably, a solution comprising carboxyaryl aldehyde dissolved in a suitable solvent for the reaction or other liquid phase form of the starting material is used. Water, lower alkyl monocarboxylic acids, benzoic acid and combinations thereof are preferred solvents for carboxyaryl aldehyde solutions, with water and aqueous lower alkyl monocarboxylic acids, especially acetic acid, being preferred. Concentration of carboxyaryl aldehyde in a solvent is not critical and can be varied as desired. Concentrations generally are low enough that the starting material is substantially dissolved and high enough for practical process operations and efficient use and handling of solvent. For practical applications, solutions comprising up to about 60 parts by weight carboxyaryl aldehyde per hundred parts by weight solution at process temperatures are suitable. In embodiments involving purification of aromatic carboxylic acids and carboxyaryl aldehyde present in minor amounts such as impurities or by-products or intermediates present from prior synthesis, carboxyaryl aldehyde can be present in reaction solutions in amounts as low as a thousand or hundreds or tens of parts per million by weight (ppmw). Preferred feed solutions thus can contain as little as about 0.001 wt % to as much as about 60 wt % or more, and more preferably about 0.01 to about 50 wt % carboxyaryl aldehyde at temperatures up to about 370° C.

Contacting the carboxyaryl aldehyde-containing starting material with hydrogen can be conducted in a batchwise, semi-continuous or continuous mode. Contacting is conducted under hydrogenation conditions, preferably including temperature and pressure effective for conversion of carboxyaryl aldehyde to hydroxyalkylaromatic acid, preferably with selectivity thereto. When using starting material in the form of a solution, temperature preferably is about 25 to about 400° C., and more preferably about 100° C. to about 370° C., more preferably to about 325° C. Temperatures of about 200 to about 300° C. are most preferred for carboxyaryl aldehyde conversions of about 95% or more. In liquid phase systems, contacting with hydrogen preferably is conducted at a pressure sufficient to maintain a liquid phase. Total pressure is at least equal to, and preferably exceeds, the sum of the partial pressures of hydrogen introduced to the process and solvent vapor(s) that boils off from the reaction mixture at the temperature of operation. Preferred pressures are at least atmospheric, and more preferably about 500 psig (˜3450 kPa), and still more preferably about 1000 psig (˜7000 kPa), to about 3000 psig (˜20800 kPa) and more preferably about 1500 psig (˜10400 kPa). Hydrogen partial pressures preferably are about 1 psi (˜7 kPa) and more preferably about 10 psi (˜70 kPa) to about 1000 psi (˜6890 kPa) more preferably 500 psi (˜3450 kPa). Residence times for contacting feed material with hydrogen in the presence of catalyst are not critical.

Conversion of carboxyaryl aldehydes according to the invention is conveniently carried out in a suitable reaction zone to which feed materials, catalyst and hydrogen together with other materials that may be used or present can be suitably added, contacted and maintained under reaction conditions, and from which a reaction mixture can be withdrawn or components thereof separated. Any suitable reaction zone can be employed, common examples being interior volumes of stirred tank, pipe, slurry, bubble column or other suitable reactor configurations. The reactor is capable of withstanding temperatures and pressures under which hydrogenation is conducted and the corrosive nature of the acidic or oxygenated reactants and products. Suitable reactors include fixed bed reactors as well as those adapted for operation with stirred or fluidized catalyst. Staged and segmented reaction zones and reactor combinations also are suitable.

Hydrogen used in the process is dihydrogen and is conveniently used in gaseous form. Non-gaseous species such as formic acid and formic acid salts that liberate dihydrogen under process conditions also may be used.

Catalysts according to the invention comprise iridium or rhodium. Supported catalysts comprising iridium or rhodium and a support material are preferred. Catalysts according to embodiments of the invention can comprise one or more additional metal or metals. Preferred catalysts comprise palladium in addition to one or both of iridium and rhodium. Nickel, copper, zinc, rhodium and combinations thereof or combinations with palladium may also provide beneficial performance.

Metal loadings for supported catalysts are not critical. Practical loadings range from about 0.1 wt % to about 10 wt % based on total weight of support and catalyst metal or metals. Preferred catalysts contain about 0.1 to about 5 wt % and more preferably about 0.2 to about 3 wt % metal(s).

Supported catalysts or components used according to the invention comprise support materials which can be in any form but preferably comprise solid particulates, such as powder, particles, pellets, granules, spheres (including microspheres), porous particles, nanotubes, colloidal and non-colloidal powders and the like. Suitable support materials include carbon, silicon carbide and refractory metal oxides such as silica, alumina, cerium oxide, silica-alumina, titania and zirconia. Preferred supports maintain physical integrity and metal loadings for suitable performance in use, including exposures to process conditions and manipulative steps. Preferred supports include carbons and metal oxides such as alpha alumina, silicas, cerium oxide and titania, including rutile, anatase and combined forms thereof. Zeolite supports are also useful but may benefit from additional stabilization for use according to the invention. Other supports which may be suitable include high strength, acid-stable silicon carbides, zirconia, gamma alumina and zinc oxide. Examples of commercially available carbon supports have BET surface areas of about one or even a fractional square meter per gram to about 1600 m²/g. Surface areas of metal oxide supports range from about 1 m²/g in the case of rutile titanias to about 500 m²/g for silicas.

Supported compositions comprising iridium or rhodium or combinations additionally comprising one or more additional metals can be prepared by any suitable method. Typically, support particles, such as pellets, granules, extrudate or other solid form suited to the manner and conditions of intended use are contacted with one or more solution or solutions of catalyst metal compound or compounds in water or another solvent that is inert to the support and easily removed, after which the solvent is removed, such as by drying at ambient or elevated temperature. For preparations in which two or more metals are used, a single solution of all catalyst metal salts or compounds can be employed as can concurrent or sequential impregnations using solutions of individual catalyst metal salts or combinations. Suitable catalyst metal compounds for support preparations are well known and include nitrates and chlorides, specific examples being iridium acetate, iridium(III) acetylacetonate, iridium(III) chloride and rhodium(III) acetate, all of which are water-soluble. Hexa(acetato)-mu-oxotris(aqua)trirhodium(III) acetate is also suitable and can be used in solid form or in aqueous solution. Palladium chloride and palladium nitrate are examples of useful salts for preparation of palladium-containing catalysts.

Incipient wetness (dry) impregnation techniques, in which a support is contacted with a solution of catalyst metal(s) compound(s) in an amount that just wets the support and the resulting wetted support is dried, are suited to manufacture of the catalysts. Eggshell impregnations in which catalyst metal particles form a thin, continuous or discontinuous layer or coating on support surfaces are also suitable. For carbon supports, eggshell impregnations, such as those with catalyst metal(s) dispersed predominantly on support surfaces, e.g., in the outermost 10 to 20% of the volume of supported catalyst particles, are preferred in some embodiments. So-called egg yolk, egg white and uniform dispersions also are contemplated. Other techniques, such as spraying a solution of catalyst metal compound onto the support also are suitable, as are excess solution methods such as wet impregnation, soaking or dipping using metal solution volumes exceeds pore volume of the support.

Post-treatments, such as high temperature calcinations in the presence of air or nitrogen, and reduction with hydrogen also can be used if desired and may yield catalysts with advantages or characteristics of interest.

Catalysts used according to the invention can provide high conversions of carboxyaryl aldehydes to hydrogenated derivatives with selectivity to hydroxyalkylaryl monocarboxylic acids. Products of the invented process thus comprise hydroxyalkylaryl monocarboxylic acid and typically also include alkylaryl monocarboxylic acid, aromatic monocarboxylic acid and aromatic dicarboxylic acids. Conversion of the carboxyaryl aldehyde starting material can range from a few percents to essentially complete conversion, depending on factors such as reaction temperature, residence time and specific catalyst composition. Conversions to hydrogenated derivatives preferably range from at least 80%, or more preferably 90% to as high as 95-100%. In embodiments using catalysts comprising iridium or rhodium and palladium, conversion of carboxyaryl aldehyde is preferably at least 95%. Selectivity in preferred embodiments is such that the mole ratio of hydroxyalkylaromatic monocarboxylic acid in a reaction product of the process to carboxyaryl aldehyde starting material exceeds that when using catalyst in which the hydrogenation metal is any of iridium, rhodium or palladium alone, other things, including carboxyaryl aldehyde conversion, being equal. More preferably, conversion of carboxyaryl aldehyde using catalyst comprising iridium or rhodium in combination with palladium is such that the mole ratio of hydroxyalkylaromatic monocarboxylic acid in a reaction product of the process to carboxyaryl aldehyde starting material is at least about 0.25:1 and especially at least about 0.3:1. Theoretically, the mole ratio has an upper limit of 1:1; practically it is up to about 0.85:1 and more typically up to about 0.65:1.

In embodiments in which catalyst comprising iridium or rhodium and palladium is used for hydrogenating 4CBA, high conversions of the carboxyaryl aldehyde are achieved with selectivity to pHMBA. Such results are achieved with or without aromatic carboxylic acid present during contacting. Preferably, pHMBA yield exceeds that when using catalysts in which the hydrogenation metal is iridium, rhodium or palladium alone, other things being equal. More preferably, conversion of 4CBA using catalyst comprising iridium or rhodium and palladium is such that the mole ratio of product pHMBA to 4CBA in the feed is at least about 0.25:1 and more preferably at least about 0.35:1. Mole ratios of pHMBA to the sum of pTOL and pHMBA preferably are at least about 0.3:1 and more preferably about 0.33:1 to about 0.85:1.

Hydroxyalkylaryl monocarboxylic acid is recovered from the reaction mixture by any suitable means. Solubility of the hydroxyalkyaryl monocarboxylic acid in aqueous solvents exceeds that of the carboxyaryl aldehyde starting material as well as solubilities of other converted products such as alkylaryl monocarboxylic acid, aryl monocarboxylic acid and dicarboxylic acids. Accordingly, the hydroxyalkyaryl monocarboxylic acid can be conveniently separated from other products by solid-liquid separation techniques such as crystallization. Taken together with reduced generation of alkylaryl monocarboxylic acid relative to hydroxyalkylaromatic monocarboxylic acid as a result of selectivity to the latter, solubility of the hydroxyalkylaryl monocarboxylic acid in aqueous or other solvents can allow for ambient or lower pressure separations than conventionally employed, thereby enabling use of simplified separation equipment and techniques. In processes according to the invention in which contacting the carboxyaryl aldehyde-containing starting material with hydrogen in the presence of catalyst comprising iridium or rhodium is conducted in aqueous solution, the hydroxyalkyaryl product preferably is recovered from the reaction solution by decreasing temperature of the reaction mixture, reducing pressure, or both, to facilitate separation of more easily crystallized by-products, such as alkylaryl monocarboxylic acids and aromatic carboxylic acids, as solids while hydroxyalkylaryl monocarboxylic acid is retained in solution in the liquid reaction mixture or other aqueous solvent used for the recovery. Crystallization temperatures preferably range from about 20° C. and more preferably 90° C. to about 200° C. and more preferably 175° C. Temperature is conveniently reduced by flashing or otherwise reducing pressure on the reaction mixture.

In embodiments in which carboxyaryl aldehyde is hydrogenated in the presence of aromatic carboxylic acid, for example as in purification of a crude or impure product comprising aromatic carboxylic acid and by-product carboxyaryl aldehyde, preferred catalyst compositions comprise iridium or rhodium and palladium. Iridium or rhodium and palladium are present in amounts such that the catalyst is active for conversion of carboxylaryl aldehyde to hydrogenated derivatives with selectivity to hydroxyalkylaromatic monocarboxylic acid. Mole ratios of iridium, rhodium or a combination thereof to palladium, each calculated as metal, may range from about from 1:100 to 100:1. Preferred catalysts for use according to the invention comprise iridium or rhodium and palladium in mole (atom) ratios of about 1:1 to about 1:100 and more preferably about 1:5 to about 1:75. Preferred catalysts in some embodiments comprise about 1 mole iridium or rhodium, and preferably iridium, to about 10 to about 50 moles palladium. Supported catalyst compositions preferably comprise iridium or rhodium and palladium supported on a support comprising carbon, and more preferably carbon having a surface area of about 100-1600 m²/g. Especially preferred supports comprise coconut shell charcoals with BET surface area of about 700-1400 m²/g. Catalysts having iridium, rhodium or a combination thereof and palladium or other additional metal(s) supported on the same support are most preferred although catalysts with the metals supported on different support compositions or partially supported on supports differing in composition or properties may be used. For such applications, catalyst is most preferably used in particulate form, for example as pellets, extrudate, spheres or granules, although other solid forms also are suitable.

In the purification of aromatic carboxylic acids having aromatic aldehyde impurities, particle size of the catalyst for fixed bed use preferably is selected such that reaction rates are not significantly adversely affected by mass transfer limitations, but a bed of catalyst particles is easily maintained in a suitable reactor for the process and permits flow of a liquid phase reaction solution or mixture comprising aromatic carboxylic acid and carboxyaryl aldehyde dissolved in aqueous solvent through the bed without undesirable pressure drop. Preferably, catalyst particles pass through a 2-mesh screen but are retained on a 24-mesh screen (U.S. Sieve Series) and more preferably pass through a 4-mesh screen but are retained on a 12-mesh and, most preferably, 8-mesh screen.

Contacting the carboxyaryl aldehyde-containing starting material with hydrogen in the presence of aromatic carboxylic acid according to these embodiments of the invention is conducted at elevated temperature and pressure. Temperature preferably ranges from about 180 to about 370° C., with about 200 to about 325° C. being more preferred. Temperatures in the upper portion of the range, such as about 275 to about 315° C. are preferred for hydrogenation of 4CBA in purification of terephthalic acid, while lower temperatures, such as about 190 to about 245° C. are most preferred for purification of isophthalic acid by hydrogenation of 3CBA. Contacting with hydrogen preferably is conducted under pressure sufficient to maintain a liquid phase reaction solution or mixture in the reaction zone. Total pressure is at least equal to, and preferably exceeds, the sum of the partial pressures of hydrogen introduced to the process and solvent vapor(s) that boils off from the reaction mixture at the temperature of operation. Preferred pressures are about 350 psig (˜2510 kPa), and more preferably about 400 psig (˜2860 kPa), to about 2000 psig (˜13900 kPa), more preferably about 1500 psig (˜10400 kPa). Total pressure of about 1000 to about 1500 psig (˜7000-10400 KPa) is most preferred in hydrogenation of 4CBA for purification of terephthalic acid according to the invention and about 350 to about 500 psig (˜2510-3550 kPa) is most preferred for hydrogenation of 3CBA in purification of isophthalic acid.

A preferred reactor configuration for fixed bed operation for hydrogenating a feed comprising carboxyaryl aldehyde and aromatic carboxylic acid or for other hydrogenations of the carboxyaryl aldehyde in the presence of aromatic carboxylic acid is a cylindrical reactor with a substantially central axis that is vertically disposed when the reactor is in use. Upflow and downflow reactors can be used. Catalyst typically is present in the reactor in one or more fixed beds of particles maintained with a mechanical support for holding the particles in the bed while allowing relatively free passage of reaction solution therethrough. A single catalyst bed is often preferred although multiple beds of the same or different catalyst or a single bed layered with different catalyst compositions, for example, with respect to particle size, catalyst metals or metal loadings, or with catalyst and other materials such as abrasives for protecting physical integrity of the catalyst, also can be used. Mechanical supports in the form of flat mesh screens or a grid formed from appropriately spaced parallel wires are suitable. Examples of other useful catalyst retaining means include tubular screens and perforated plate supports. The mechanical support for the catalyst bed is constructed of materials that suitably resist corrosion due to contact with the acidic reaction solution, and are strong enough to efficiently retain the catalyst bed. Supports for catalyst beds typically have openings of about 1 mm or less and are constructed of metals such as stainless steel, titanium or Hastelloy C.

In such embodiments, a solution of impure aromatic carboxylic acid comprising carboxyaryl aldehyde preferably is added to the reactor vessel at elevated temperature and pressure at a position at or near a top portion of the reactor vessel and the solution flows downwardly through a catalyst bed contained in the reactor vessel in the presence of hydrogen gas, wherein the aldehyde substituents of the carboxyaryl aldehyde are hydrogenated to alcohol. In such embodiments, the reactor may be operated in several modes. In one mode, a predetermined liquid level can be maintained in the reactor and, for a given reactor pressure, hydrogen can be fed at a rate sufficient to maintain the predetermined liquid level. The difference between the actual reactor pressure and the vapor pressure of the vaporized reaction solution present in the reactor head space is the hydrogen partial pressure in the head space. Alternatively, hydrogen can be fed mixed with an inert gas such as nitrogen or water vapor, in which case the difference between the actual reactor pressure and the vapor pressure of the vaporized reaction solution present is the combined partial pressure of hydrogen and the inert gas admixed therewith. In the latter case the hydrogen partial pressure may be calculated from the known relative amounts of hydrogen and inert gas present in the admixture.

In another operating mode, the reactor can be filled with the liquid reaction mixture so that there is no reactor vapor space. In such an embodiment, the reactor is operated as a hydraulically full system with dissolved hydrogen being fed to the reactor by flow control. The concentration of hydrogen in solution may be modulated by adjusting hydrogen flow rate to the reactor. If desired, a pseudo-hydrogen partial pressure value may be calculated from the solution hydrogen concentration which, in turn, may be correlated with hydrogen flow rate to the reactor.

When operating such that process control is effected by adjusting hydrogen partial pressure, hydrogen partial pressure in the reactor is preferably about 10 to about 200 psi (about 69-1380 kPa) or higher, depending on pressure rating of the reactor, choice of starting material, activity and age of the catalyst and other considerations known to persons skilled in the art. In the operating mode in which process control is affected by direct adjustment of hydrogen concentration in the feed solution, the latter usually is less than saturated with respect to hydrogen and the reactor itself is hydraulically full. Thus, an adjustment of the hydrogen flow rate to the reactor results in the desired control of hydrogen concentration in the solution.

Space velocity in such embodiments, expressed as weight of aromatic carboxylic acid starting material comprising carboxyaryl aldehyde per weight of catalyst per hour, is typically about 1 hour⁻¹ to about 25 hour⁻¹, and preferably about 2 hours⁻¹ to about 15 hours⁻¹. Residence time of the liquid stream comprising feed material in the catalyst bed varies with space velocity.

After contacting carboxyaryl aldehyde with hydrogen in the presence of unsubstituted aromatic carboxylic acid and the iridium or rhodium and palladium-containing catalyst according to such embodiments of the invention, a liquid reaction mixture comprising hydroxyalkyaryl monocarboxylic acid, e.g., a hydroxymethylbenzoic acid such as mHMBA or pHMBA, and the aromatic carboxylic acid, such as isophthalic acid or terephthalic acid, preferably is cooled to separate purified, solid aromatic carboxylic acid from the liquid reaction mixture, leaving a liquid mixture, sometimes also referred to as a mother liquor, in which hydroxyalkyaryl monocarboxylic acid and other soluble hydrogenated species that may be present remain dissolved. Separation is commonly achieved by cooling to a crystallization temperature, which is sufficiently low for crystallization of the purified aromatic carboxylic acid to occur, thereby producing solid product within the liquid phase. The crystallization temperature is sufficiently high that impurities and their reduction products resulting from hydrogenation remain dissolved in the liquid phase. Crystallization temperatures for aromatic carboxylic acid product generally range up to 180° C. and preferably up to about 150° C. Crystallization temperatures for terephthalic acid are generally higher than for isophthalic acid. In continuous operations, separation normally comprises removing the hydrogenated reaction solution from the hydrogenation reactor and crystallization of aromatic carboxylic acid in one or more crystallization vessels. When conducted in a series of stages or separate crystallization vessels, temperatures in the different stages or vessels can be the same or different and preferably decrease from each stage or vessel to the next. In preferred embodiments of the invention, conversion of carboxyaryl aldehyde such as 4CBA and selectivity to hydroxyalkylaryl carboxylic acid, e.g., pHMBA, are sufficiently high and alkylaryl carboxylic acid, such as pTOL, which has a greater propensity than pHMBA to co-crystallize with terephthalic acid, is present in amounts small enough that one or more crystallization steps, and most preferably a final step or steps can be carried out at low or even ambient pressures and more preferably about 0 to about 15 psig ((˜100-200 kPa). Thereafter, crystallized, purified aromatic carboxylic acid product is recovered from the mother liquor. Recovery of the crystallized purified product is commonly conducted by centrifuging or by filtration.

Reactor and catalyst bed configurations and operating details and crystallization and product recovery techniques and equipment useful according to the invention are described in further detail in U.S. Pat. No. 4,629,715, U.S. Pat. No. 4,892,972, U.S. Pat. No. 5,175,355, U.S. Pat. No. 5,354,898, U.S. Pat. No. 5,362,908 and U.S. Pat. No. 5,616,792 which are incorporated herein by reference.

When practicing the invention for purification or crude or impure aromatic carboxylic acids, the aromatic carboxylic acids generally contain one or more aromatic nuclei and 1 to about 4 carboxylic acid groups. Examples include benzoic acid, phthalic acid, terephthalic acid, isophthalic acid, t-butyl isophthalic acid, trimesic acid, trimellitic acid, and naphthalene dicarboxylic acids. Preferred aromatic carboxylic acids are dicarboxylic acids with a single aromatic ring and especially terephthalic acid. In commercial practice, these acids are often obtained by metal-catalyzed oxidation of feed materials comprising aromatic compounds with oxidizable substituents, such as toluene, xylenes, trimethylbenzenes and dimethyl and diethyl naphthalenes.

The impure aromatic carboxylic acid compositions also comprise carboxyaryl aldehyde. The impure aromatic carboxylic acid may also comprise one or more other impurities. In the case of an impure aromatic carboxylic acid comprising a crude product obtained by liquid phase oxidation of feed materials comprising aromatic compounds with oxidizable substituent groups, impurities comprise oxidation by-products or intermediates. In the case of a crude terephthalic acid product obtained by liquid phase oxidation of feed materials such as p-xylene, common intermediates or by-products of the oxidation comprise 4CBA and may also include one or more of pHMBA, pTOL, p-dihydroxymethylbenzene, tolualdehyde, terephthalaldehyde, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene.

Amounts of carboxyaryl aldehyde present in the impure aromatic carboxylic acids to be treated according to this embodiment of the invention vary. Generally, any amount of such impurities may be present without hindering effectiveness of the invention. Aromatic carboxylic acids as obtained in liquid phase oxidations of alkyl aromatic feed materials often contain as much as 1 to 2 wt % impurities, with about 500 ppmw up to about 1 wt % being more common in commercial operations. Preferred catalysts for use in such embodiments of the invention comprise about 5 and more preferably about 10 to about 75, more preferably about 50, moles palladium per mole of iridium, rhodium or combination thereof. Iridium is preferred over rhodium. Best results in such embodiments are attained with catalysts supported on carbon; titania and acid-stable silicon carbide supports also give good results.

In a more specific embodiment of the invention, the impure aromatic carboxylic acid product to be purified according to the invention comprises a crude aromatic carboxylic acid product obtained by liquid phase oxidation of a feed material comprising at least one aromatic compound with substituents oxidizable to carboxylic acid groups. Such oxidations are commonly conducted in a liquid phase reaction mixture comprising a monocarboxylic acid solvent and water using oxygen as the oxidant and in the presence of a heavy metal catalyst.

Feed materials for manufacture of such crude aromatic acid products generally comprise an aromatic hydrocarbon substituted with at least one group that is oxidizable to a carboxylic acid group. The oxidizable substituent or substituents can be an alkyl group, such as a methyl, ethyl or isopropyl group. The substituents also can include one or more groups already containing oxygen, such as a hydroxyalkyl, formyl or keto group. The substituents can be the same or different. The aromatic portion of feedstock compounds can be a benzene nucleus or it can be bi- or polycyclic, such as a naphthalene nucleus. The number of oxidizable substituents on the aromatic portion of the feedstock compound can be equal to the number of sites available on the aromatic portion but is generally less than all such sites, preferably 1 to about 4 and more preferably 1 to 3. Examples include toluene, ethylbenzene, o-xylene, p-xylene, m-xylene, 1-formyl-4-methylbenzene, 1-hydroxymethyl-4-methylbenzene, 1,2,4-trimethyl-benzene, 1-formyl-2,4-dimethylbenzene, 1,2,4,5-tetramethylbenzene, and alkyl-, acyl-, formyl- and hydroxymethyl-substituted naphthalenes such as 2,6- and 2,7-dimethylnaphthalenes, 2-acyl-6-methylnaphthalene, 2,6-diethylnaphthalene, 2-formyl-6-methylnaphthalene and 2-methyl-6-ethylnaphthalene.

For manufacture of a crude aromatic acid product by oxidation of corresponding aromatic feed pre-cursors, e.g., manufacture of isophthalic acid from meta-disubstituted benzenes, terephthalic acid from para-disubstituted benzenes, trimellitic acid from 1,2,4-trisubstituted benzenes, naphthalene dicarboxylic acids from disubstituted naphthalenes, it is preferred to use relatively pure feed materials, and more preferably, feed materials in which content of the precursor corresponding to the desired acid is at least about 95 wt. %, and more preferably at least 98% or even higher. A preferred aromatic feed for use to manufacture terephthalic acid comprises para-xylene. A preferred feed for isophthalic acid comprises meta-xylene.

Oxidant used for the liquid phase oxidations comprises molecular oxygen which is preferably in gaseous form. Air is conveniently used as a source of oxygen. Oxygen-enriched air, pure oxygen and other gaseous mixtures comprising at least about 10% molecular oxygen also are useful.

Catalysts used in such oxidations comprise materials that are effective to catalyze oxidation of the aromatic hydrocarbon feed to aromatic carboxylic acid. Preferably, the catalyst is soluble in the liquid oxidation reaction body to promote contact among catalyst, oxygen and liquid feed; however, heterogeneous catalyst or catalyst components may also be used. Typically, the catalyst comprises at least one heavy metal component such as a metal with atomic weight in the range of about 23 to about 178. Examples include cobalt, manganese, vanadium, molybdenum, chromium, iron, nickel, zirconium, cerium or a lanthanide metal such as hafnium. Preferably, catalyst comprising one or both of cobalt and manganese is used. Soluble forms of these metals include bromides, alkanoates and bromoalkanoates; specific examples include cobalt acetate and bromide, zirconium acetate and manganese acetate and bromide.

For some catalysts, and particularly those comprising cobalt, manganese or combinations thereof, a promoter is also used. The promoter promotes oxidation activity of the catalyst metal(s), preferably without generation of undesirable types or levels of by-products, and is preferably used in a form that is soluble in the liquid reaction mixture. Preferably the promoter comprises bromine, including elemental, ionic and organic forms. Examples include Br₂, HBr, NaBr, KBr, NH₄Br, bromobenzenes, benzyl-bromide, bromo acetic acid, dibromo acetic acid, tetrabromoethane, ethylene dibromide and bromoacetyl bromide. Other suitable promoters include aldehydes and ketones, such as acetaldehyde and methyl ethyl ketone. Bromine-free catalysts such as disclosed in WO 2007/133978 and WO 2007/133973, both published 22 Nov. 2007, and WO 2008/137491 published 13 Nov. 2008 also are suitable.

A solvent for the feed material, soluble catalyst materials and promoter, if used, is desirably used in the process. Solvents comprising an aqueous carboxylic acid, and especially a lower alkyl (e.g., C₁₋₆) monocarboxylic acid, are preferred because they tend to be only sparingly prone to oxidation under typical oxidation reaction conditions and can enhance catalytic effects in the oxidation. Specific examples of suitable carboxylic acids include acetic acid, propionic acid, butyric acid, benzoic acid and mixtures thereof. Water is useful in some embodiments. Co-solvent materials which oxidize to monocarboxylic acids under oxidation reaction conditions also can be used as is or in combination with carboxylic acids with good results.

Proportions of the feed, catalyst, oxygen and solvent are not critical and vary not only with choice of feed materials and intended product but also choice of process equipment and operating factors. Solvent to feed weight ratios suitably range from about 1:1 to about 30:1. Oxygen typically is used in at least a stoichiometric amount based on feed but not so great that unreacted oxygen escaping from the liquid body to the overhead gas phase forms a flammable mixture with other components of the gas phase. Catalysts suitably are used in weights providing about 100 to about 3000 ppm catalyst metal(s) based on feed weight. Promoter concentrations also generally range from about 100 to about 3000 ppm based on weight of the liquid feed, with about 0.1 to about 2 milligram-atoms promoter suitably used per milligram-atom catalyst metal.

Oxidation of aromatic feed materials to crude product comprising aromatic acid and by-product carboxyaryl aldehyde is conducted under oxidation reaction conditions. Temperatures in the range of about 120 to about 250° C. are generally suitable, with about 150 to about 230° C. preferred. Pressure in the reaction vessel is at least high enough to maintain a substantial liquid phase comprising feed and solvent in the vessel. Generally, pressures of about 5 to about 35 kg/cm² gauge are suitable, with preferred pressures for particular processes varying with feed and solvent compositions, temperatures and other factors. Solvent residence times in the reaction vessel can be varied as appropriate for given throughputs and conditions, with about 20 to about 150 minutes being generally suited to a range of processes. For processes in which the aromatic acid product is substantially soluble in the reaction solvent, such as in the manufacture of trimellitic acid by oxidation of psuedocumene in acetic acid solvent, solid concentrations in the liquid body are negligible. In other processes, such as oxidation of xylenes to isophthalic or terephthalic acids, solids contents can be as high as about 50 wt. %. Preferred conditions and operating parameters vary with different products and processes and can vary within or outside preferred ranges.

Crude aromatic carboxylic acid products of such liquid phase oxidation processes include by-product carboxyaryl aldehyde and commonly also include other intermediates and by-products. Examples of those intermediates and by-products include aldehydes and ketones such as carboxybenzaldehydes, fluorenones and dicarboxyanthroquinones as described above. Impurities levels up to 2 wt % or even higher, depending on feed materials, operating parameters and process efficiency, are not uncommon and can be enough to affect product quality of the desired carboxylic acid product or downstream products thereof.

In a particular embodiment, the invention is used for the manufacture of a purified aromatic carboxylic acid comprising terephthalic acid from a crude aromatic carboxylic acid product comprising terephthalic acid and by-product 4CBA obtained by liquid phase oxidation of an aromatic hydrocarbon feed comprising para-xylene or its partially oxidized derivatives or combinations thereof. Acetic acid or aqueous acetic acid is a preferred solvent, with a solvent to feed ratio of abut 2:1 to about 5:1 being preferred. The catalyst preferably comprises cobalt, manganese or a combination thereof, and a source of bromine soluble in the solvent is preferably used as promoter. Cobalt and manganese preferably are used in amounts providing about 100 to about 800 ppmw based on feed weight. Bromine preferably is present such that the atom ratio of bromine to catalyst metal is about 0.1:1 to about 1.5:1.

Oxygen-containing gas is provided to the liquid phase reaction mixture at a rate effective to provide at least about 3 moles molecular oxygen per mole of aromatic feed material and, in conjunction with removal of reactor off-gases, such that unreacted oxygen in the vapor space above the liquid reaction body is below the flammable limit. With air as the oxygen source, the limit is about 8 mole % measured after removal of condensable compounds.

Oxidation preferably is conducted at temperatures of about 160 to about 225° C. under pressure of about 5 to about 20 kg/cm² gauge. At those conditions, contact of the oxygen and feed material in the liquid body results in formation of solid terephthalic acid crystals, typically in finely divided form. Solids content of the boiling liquid slurry typically ranges up to about 40 wt % and water content typically is about 5 to about 20 wt % based on solvent weight. Boiling of the liquid body for control of the reaction exotherm causes volatilizable components of the liquid body, including solvent and water of reaction, to vaporize. Unreacted oxygen and vaporized liquid components escape from the liquid into the reactor space above the liquid. Other species, for example nitrogen and other inert gases that are present if air is used as an oxygen source, carbon oxides, and vaporized by-products, e.g., methyl acetate and methyl bromide, also may be present in the overhead vapor.

Crude product from the oxidation is separated from the liquid reaction mixture, typically by crystallization at reduced temperature and pressure, and the resulting solid is recovered by filtration or centrifuging. The recovered crude terephthalic acid comprises 4CBA, typically in amounts ranging from about 500 to about 5000 ppmw. Purification of the crude product according to the invention typically reduces levels of 4CBA in the purified terephthalic acid to below about 100 ppmw, preferably about 25 ppmw or less.

Embodiments and aspects of the invention are described further in the examples, which are presented for purposes of illustration, not limitation.

### EXAMPLES

### General Procedures for Examples 1 and 2

Catalysts in Examples 1 and 2, including Catalysts 1-4 and Comparative A, were supported catalysts prepared by an incipient wetness technique. Aqueous solutions of iridium acetate, palladium nitrate and rhodium acetate, each from W. C. Heraeus, and a granular, coconut shell carbon from Norit designated GCN 3070 were used. The carbon had pore volume of 0.60 mL/g determined by water absorption.

Impregnations were conducted by placing in glass vials weighed amounts of the carbon, which had been dried at 120° C. in air before weighing, and adding to the bottles volumes of one or both of the metals salt solutions equal to the pore volumes of the support samples. After adding the solutions to the carbon samples the bottles were tumbled on a rolling bench for at least 1 hour to evenly spread excess moisture on the outside of the carbon particles and allow the solution(s) to penetrate the pores of the carbon. After tumbling the catalyst samples were dried in air for 2 hours at 110° C., calcined under a nitrogen flow at 100 mL/min for 2 hours at 300° C., reduced in a flow of 7% hydrogen in nitrogen at 100 mL/min for 5 hours at 250° C., and ground to powder using a mechanical grinding mill. Catalysts were prepared with iridium, rhodium and palladium weight percents and mole ratios as reported in the following table.

| | Metal(s) & Loading(s) | Ir/Pd/Rh mole |
|---|---|---|
| Catalyst | (wt % Ir/Pd/Rh) | ratio |
| Comparative A | 0/0.50/0 | 0 |
| 1 | 0.90/0.50/0 | 1:1:0 |
| 2 | 1.8/0.50/0 | 1:2:0 |
| 3 | 0.45/0.50/0 | 2:1:0 |
| 4 | 0/0.50/0.48 | 0:1:1 |

Catalytic hydrogenation experiments were conducted using parallel magnetically-stirred, stainless steel batch reactors with a volume of 50 mL each. The reactors were fitted with Teflon insert liners. Reactors were charged with solid catalyst, 15 mL deionized water and about 15 mg (about 0.1 mmol) 4-carboxybenzaldehyde at room temperature. In Example 2 the charge to the reactor also included about 1.5 g (about 9 mmol) terephthalic acid. The reactors were then purged with nitrogen, tested for leaks by pressuring to 30 bar with nitrogen followed by releasing the pressure and pressurizing with hydrogen to 10 bar, heated to 275° C. over 30-45 minutes, held at 275-282° C. for 20 minutes, and then allowed to cool. During heating back pressure regulators with which the reactors were equipped were set at 90 bar, effectively sealing the reactors.

After cooling, reactor contents were diluted with dimethyl sulfoxide (“DMSO”) to dissolve the organic solids. The solutions were analyzed for 4CBA, terephthalic acid (TA), pTOL, benzoic acid (BA), and pHMBA by high-pressure liquid chromatography (“HPLC”).

### Example 1

Hydrogenation reactions of 4CBA in water were performed using Comparative A and Catalysts 2 and 3. Results are reported in Table 1.

TABLE 1

| Catalyst | Comparative A | 2 | 3 |
|---|---|---|---|
| Metal(s) and Ir/Pd Mole | Pd (0:1) | Ir/Pd (2:1) | Ir/Pd (1:2) |
| Ratio | | | |
| Catalyst Charge (mg) | 12.0 | 10.9 | 12.1 |
| 4CBA charge (mg) | 0.104 | 0.101 | 0.110 |
| 4CBA Conversion (%) | 99.7 | 100 | 100 |

Selectivities (mole % based on sum of pHMBA, pTOL, BA and TA)

| | | | |
|---|---|---|---|
| pHMBA | 8 | 32 | 53 |
| pTOL | 79 | 25 | 27 |
| BA | 5 | 30 | 9 |
| TA | 9 | 13 | 11 |
| Sum | 101* | 100 | 100 |
| pHMBA/pTOL Mole Ratio | 0.09 | 1.3 | 1.9 |

*This sum exceeds 100 mole % due to rounding.

All catalysts exhibited essentially 100% conversion of 4CBA but they differed in selectivity. Catalysts 2 and 3, containing iridium and palladium, showed much higher selectivity for pHMBA than Comparative catalyst A, which contained only palladium and yielded primarily pTOL. The iridium and palladium-containing catalysts both yielded more pHMBA than pTOL.

### Example 2

Hydrogenation reactions of 4CBA in water were performed as described above but in the presence of TA using Comparative Catalyst A, Catalysts 2, 3 and 4, and, for comparative purposes, a commercial 0.5 wt % Pd/carbon catalyst identified as BASF “Type D” that had been dried and ground to powder.

TABLE 2

| | Catalyst No. | | | | | | | |
|---|---|---|---|---|---|---|---|---|
| | Type D | Comparative A | | | 2 | 2 | 3 | 4 |
| Metal(s) | Pd | Pd | Pd | Pd | Ir/Pd | Ir/Pd | Ir/Pd | Pd/Rh |
| Catalyst Charge | 5 | 65.9 | 10.6 | 12.3 | 9.0 | 11.1 | 12.4 | 11.3 |
| (mg) | | | | | | | | |
| 4CBA charge | 0.101 | 0.099 | 0.099 | 0.097 | 0.100 | 0.105 | 0.103 | 0.102 |
| (mmol) | | | | | | | | |
| TA charge | 9.065 | 9.021 | 9.072 | 8.998 | 9.015 | 9.136 | 9.137 | 8.955 |
| (mmol) | | | | | | | | |
| 4CBA | 96.9 | 97.5 | 98.0 | 98.7 | 98.8 | 98.8 | 97.6 | 96.6 |
| Conversion (%) | | | | | | | | |

Yields (mmol)

| | | | | | | | | |
|---|---|---|---|---|---|---|---|---|
| pHMBA | 0.017 | 0.013 | 0.026 | 0.041 | 0.062 | 0.059 | 0.058 | 0.034 |
| p-TOL | 0.075 | 0.053 | 0.062 | 0.052 | 0.014 | 0.018 | 0.017 | 0.020 |
| BA | 0.006 | 0.016 | 0.002 | 0.003 | 0.018 | 0.021 | 0.010 | 0.027 |
| Product Mole | 0.18 | 0.20 | 0.30 | 0.44 | 0.81 | 0.76 | 0.77 | 0.63 |
| Ratios: pHMBA/ | | | | | | | | |
| (pHMBA + pTOL) | | | | | | | | |

As seen from the table, the reactions proceeded to 97-99% 4CBA conversion. The reactions with the Pd—Ir catalysts and the Pd—Rh catalyst yielded much higher amounts of pHMBA than pTOL whereas the comparative catalysts without Ir or Rh yielded more pTOL than pHMBA.

### General Procedures for Examples 3 and 4

Catalyst samples were prepared from stock solutions of Pd(NO₃)₂ (14 wt % Pd) and iridium acetate (5 wt % Ir, in 50% acetic acid solution) obtained from Johnson Matthey and from hexa(acetato)-mu-oxotris(aqua)trirhodium(III) acetate ([Rh₃(OOCCH₃)₆-μ-O(H₂O)₃]OAc) obtained from Alfa Aesar.

Catalytic hydrogenation experiments were conducted with the Catalysts in these examples using 435 grams crude terephthalic acid containing about 0.25 wt % 4CBA and about 0.1 wt % other impurities such as pTOL, pHMBA and benzoic acid (“BA”) and 1015 grams of water. The crude terephthalic acid and water were charged to a one-gallon titanium autoclave reactor. Reactor contents were stirred at 300 revolutions per minute and hydrogen gas at 20° C. in a volume corresponding to 0.42 moles was added to the reactor from a 300 ml vessel by lowering the vessel pressure by 500 psi (˜690 kPa). The reactor was heated to 290° C. to dissolve the terephthalic acid, after which the stirring rate was increased to 1000 revolutions per minute and the catalyst sample being tested was added to the reactor as described in more detail in the individual examples.

Liquid samples were withdrawn at various times after catalyst addition and analyzed for the following compounds: 4-carboxybenzaldehyde (4CBA), 4-hydroxymethyl benzoic acid (pHMBA), p-toluic acid (pTOL), and benzoic acid (BA). The results are presented in Tables 3 and 4. Samples were selected where 4CBA conversions were between 93 and 98% to allow comparisons of catalysts on a common 4CBA conversion basis. Selectivities were defined as the moles produced divided by the number of moles of 4CBA converted. As an example, selectivity to p-hydroxymethyl benzoic acid (pHMBA) was determined as follows:

$$pHMBA Selectivity = \frac{moles of pHMBA produced}{initial moles 4 C B A - final moles 4 C B A} .$$

### Example 3

Catalysts 5-8 and Comparative B were prepared from 30-70 mesh carbon particles (Norit GCN3070) which had been dried in air at 110° C. in an oven for at least 2 hours prior to use and stored in a sealed container in a desiccated environment until use. Water absorption of the carbon, measured by addition of water to the incipient wetness point, was determined to be 1.0 cc water/gram carbon.

For catalyst preparations, portions of the dried support were impregnated at room temperature with the Pd(NO₃)₂ solution or a solution containing both dissolved Pd(NO₃)₂ and iridium acetate. The solutions contained amounts of metals to yield finished catalysts with 0.5 wt % Pd and iridium contents reported in Table 3. Deionized water contents were such that solution volumes were equal to the water absorption volume of the quantities of carbon support being impregnated. Impregnations were performed by adding metal solutions to the carbon slowly and evenly using a pipette, with frequent mixing of the carbon during impregnations.

The impregnated materials were dried in an oven in air at 110° C. for 2 hours and were then loaded into a stainless steel tube inside a furnace. A 100 standard cm³/minute (“sccm”) flow of helium through the tube was begun. The tube was then heated to 300° C. and held at 300° C. for 2 hours under helium flow. Temperature was reduced to 200° C., helium flow was discontinued and a flow of hydrogen gas at 100 sccm was initiated. The tube was heated to 275° C. and held at that temperature under hydrogen flow for 2 hours, cooled to room temperature under helium flow, then unloaded.

In the hydrogenation trials, catalyst samples were released directly into the liquid phase reaction mixtures from a solids holding device fitted in the reactors in the headspace above the liquid level.

Results of the trials are reported in Table 3.

TABLE 3

| Catalyst | Comp B | 5 | 6 | 7 | 8 |
|---|---|---|---|---|---|
| Ir:Pd Atom Ratio | 0:100 | 1:25.1 | 1:10 | 1:1 | 2:1 |
| Ir, wt % | 0 | 0.036 | 0.09 | 0.903 | 1.807 |
| 4CBA Conversion | 96.1 | 97.1 | 95.7 | 97.4 | 97.2 |
| pHMBA Selectivity | 32.7 | 44.4 | 42.5 | 25.9 | 18.5 |
| pTOL Selectivity | 50.2 | 37.4 | 30.2 | 25.7 | 26.9 |
| BA Selectivity | 36 | 29.1 | 36.6 | 43.8 | 70.6 |
| Product Mole | 0.43 | 0.59 | 0.65 | 0.58 | 0.48 |
| Ratios: pHMBA/ | | | | | |
| (pHMBA + pTOL) | | | | | |

As can be seen from Table 3, pHMBA selectivities increased as the atom ratio of iridium to palladium decreased over the range of 1:10 to 25 and exceeded that of Comparative B in which palladium was the sole hydrogenation metal. BA selectivities also decreased over that range.

### Example 4

Catalyst samples 9-13 and Comparative C were prepared using 4-8 mesh granular carbon obtained from BASF which had been dried in air and stored in like manner to the 30-70 mesh carbon particles. Water absorption of this carbon was determined to be 0.8 cc water/gram carbon.

Portions of the dried 4-8 mesh carbon were impregnated at room temperature with either the aqueous solution of Pd(NO₃)₂, a solution containing both dissolved Pd(NO₃)₂ and iridium acetate, or a solution containing both dissolved Pd(NO₃)₂ and [Rh₃(OOCCH₃)₆-μ-O(H₂O)₃]OAc. These solutions contained appropriate amounts of metals to yield finished catalysts with 0.5 wt % Pd and the iridium or rhodium contents given in Table 4. Deionized water contents of the solutions were such that solution volumes were equal to the water absorption volume of the quantity of carbon support being impregnated. Impregnations were performed as described above.

The impregnated materials were dried in an oven in air at 110° C. for 2 hours and were loaded into a stainless steel tube inside a furnace. A flow of helium through the tube as in Examples 1 and 2 was initiated. The tube was then heated to 300° C. and held at 300° C. for 2 hours under helium flow. Temperature was reduced to 250° C., helium flow was discontinued, and a flow of hydrogen gas was initiated. The tube was heated to 275° C. and held at 275° C. under hydrogen flow for 2 hours, cooled to room temperature under helium flow and unloaded.

Catalyst samples were aged for 72 hours by heating at 290° C. in admixture with an aqueous solution containing 30 wt % terephthalic acid in a titanium basket and in the presence of hydrogen. In the hydrogenation trials, 10 ml catalyst samples were loaded into a 14 mesh, titanium wire screen basket through which water could flow freely. The sample-containing baskets were placed in the reactors above the level to which they were to be filled with liquid for the trials and, when the reactors reached temperature, screen baskets were lowered into the liquid reaction mixtures.

Results of the trials are reported in Table 4.

TABLE 4

| | Catalyst | | | | | |
|---|---|---|---|---|---|---|
| | Comp. C | 9 | 10 | 11 | 12 | 13 |
| Metals | Pd | Ir—Pd | Ir—Pd | Rh—Pd | Rh—Pd | Rh—Pd |
| Pd:Ir or Rh | 100/0 | 2 | 25 | 2 | 10 | 25 |
| Atom Ratio | | | | | | |
| Ir or Rh, wt % | 0 | 0.452 | 0.036 | 0.242 | 0.048 | 0.019 |
| 4CBA | 97.2 | 95.4 | 93.3 | 95.7 | 94.4 | 96.7 |
| Conversion | | | | | | |
| pHMBA | 24 | 34.8 | 41.9 | 32.3 | 32.7 | 30.2 |
| Selectivity | | | | | | |
| pTOL Selectivity | 83.4 | 73.9 | 59.5 | 26.6 | 33.2 | 44.2 |
| BA Selectivity | 44.4 | 48 | 37.2 | 58 | 49.4 | 48.8 |
| Product Mole | 0.24 | 0.34 | 0.44 | 0.62 | 0.55 | 0.45 |
| Ratios: pHMBA/ | | | | | | |
| (pHMBA + pTOL) | | | | | | |

As seen from Table 4, pHMBA selectivities using Catalysts 9-13 were improved over the selectivity of Comparative C. Selectivity to pHMBA increased and BA selectivity decreased as Pd:Ir atom ratios increased, similar to Catalysts 5 and 6 in Example 3.

We claim:

1. A process for producing hydroxyalkyl aryl monocarboxylic acid comprising contacting a feed comprising carboxyaryl aldehyde with hydrogen under hydrogenation reaction conditions in the presence of a catalyst comprising iridium or rhodium to form a product comprising hydroxyalkylaromatice monocarboxylic acid.

2. The process of claim 1 wherein the catalyst comprises iridium.

3. The process of claim 2 wherein the catalyst additionally comprises palladium.

4. The process of claim 1 wherein the feed comprises 4-carboxybenzaldehyde and the product comprises p-hydroxymethylbenzoic acid.

5. The process of claim 4 wherein the catalyst comprises iridium or rhodium and palladium.

6. The process of claim 5 wherein the catalyst is a supported catalyst.

7. The process of claim 5 wherein contacting the feed with hydrogen is conducted in the presence of aromatic carboxylic acid.

8. The process of claim 1 wherein contacting the feed with hydrogen is conducted in the presence of aromatic carboxylic acid.

9. The process of claim 1 wherein the feed comprises 4CBA and is contacted with hydrogen in the presence of terephthalic acid.

10. The process of claim 9 wherein feed comprising 4CBA and terephthalic acid dissolved in an aqueous solvent is contacted with hydrogen in the presence of the catalyst to provide a liquid phase reaction mixture comprising p-hydroxymethylbenzoic acid and terephthalic acid and a solid comprising terephthalic acid is crystallized from the liquid phase reaction mixture.

11. The process of claim 10 wherein solid terephthalic acid and a liquid phase comprising dissolved p-hydroxymethylbenzoic are separated.

12. A process for manufacture of an aromatic carboxylic acid comprising contacting a feed comprising aromatic carboxylic acid and impurities comprising carboxyaryl aldehyde with hydrogen in the presence of a catalyst comprising palladium and iridium or rhodium under hydrogenation conditions to form a product comprising aromatic carboxylic acid and hydroxyalkylaryl monocarboxylic acid wherein the mole ratio of hydroxyalkylaryl monocarboxylic acid in the product to carboxyaryl aldehyde in the feed is at least about 0.25.

13. The process of claim 12 wherein a liquid phase comprising at least a portion of the feed is contacted with hydrogen in the presence of catalyst.

14. The process of claim 13 wherein a liquid reaction mixture resulting from contacting the feed with hydrogen in the presence of catalyst is treated to crystallize a solid comprising the aromatic carboxylic acid.

15. The process of claim 14 wherein separation comprises at least one step comprising crystallizing solid aromatic carboxylic acid from the liquid reaction mixture under pressure up to about 15 psig (200 kPa).

16. The process of claim 14 wherein the catalyst comprises about 5 to about 75 moles palladium per mole iridium or rhodium.

17. The process of claim 1 wherein the feed comprises aromatic carboxylic acid and impurities resulting from oxidation of at least one alkylarene or partially oxidized derivative thereof.

18. The process of claim 9 wherein the feed comprises terephthalic acid and 4-carboxybenzaldehyde and the product comprises terephthalic acid and p-hydroxymethylbenzoic acid in excess of p-toluic acid.

19. The process of claim 18 wherein the catalyst is a supported catalyst comprising palladium, iridium and a carbon support in which iridium and palladium are present in amounts such that the mole ratio of iridium to palladium is about 1:5 to about 1:50.

20. The process of claim 1 wherein the feed comprises isophthalic acid and 3-carboxybenzaldehyde and the product comprises isophthalic acid and m-hydroxymethylbenzoic acid.
